(19) United States
(12) Patent Application Publication
(10) Pub. No.: US 2018/0160886 A1
(43) Pub. Date: Jun. 14, 2018

(54) SPRAY CAP FOR CHROMOENDOSCOPY

(71) Applicant: THE REGENTS OF THE UNIVERSITY OF MICHIGAN, Ann Arbor, MI (US)

(72) Inventors: Shail GOVANI, Ann Arbor, MI (US); Akbar WALJEE, Ann Arbor, MI (US); Gene PARUNAK, Saline, MI (US); Steven MEINES, Ypsilanti, MI (US)

(21) Appl. No.: 15/578,043

(22) PCT Filed: Jul. 19, 2016
(86) PCT No.: PCT/US2016/042960

§ 371 Date: Nov. 29, 2017

Related U.S. Application Data
(60) Provisional application No. 62/194,482, filed on Jul. 20, 2015.

(51) Int. Cl. A61B 1/00

(52) U.S. Cl. CPC A61B 1/00137; A61M 31/005; A61B 1/00091; A61B 1/00101

(57) ABSTRACT

A spray cap for use with an endoscope. The endoscope includes a liquid channel and an open channel. The spray cap includes a body portion connectable with an endoscope having an end wall defining an obstruction portion configured to obstruct the liquid channel of the endoscope and an open portion configured to permit use of the open channel. The spray cap having an orifice system extending through the obstruction portion in fluid communication with the liquid channel to direct fluid flow from the liquid channel into a treatment volume of a patient along a modified flow pattern.

### CROSS-REFERENCE TO RELATED APPLICATIONS

This application claims the benefit of U.S. Provisional Application No. 62/194,482, filed on Jul. 20, 2015. The entire disclosure of the above application is incorporated herein by reference.

### FIELD

The present disclosure relates to chromoendoscopy and, more particularly, to a spray cap for use in chromoendoscopy.

### BACKGROUND AND SUMMARY

This section provides background information related to the present disclosure which is not necessarily prior art. This section provides a general summary of the disclosure, and is not a comprehensive disclosure of its full scope or all of its features.

Chromoendoscopy is a medical procedure where stains or dyes are applied topically at the time of endoscopy to enhance tissue characterization, differentiation, or diagnosis. It has been shown to improve visualization of pre-cancerous lesions, especially small flat lesions. Currently, a spray catheter is used to spray stains onto the mucosal lining of the intestinal tract during chromoendoscopy. Yet the limitation is that the spray catheter takes up the entire open channel (also known as biopsy channel), which prevents other procedures through the channel, such as tissue biopsy.

This new device will replace the spray catheter, and be positioned at the endoscope tip. It only covers the irrigation channel but not the open channel. The partial coverage allows stain spray and other procedures at the same time, which improves operation efficiency.

One potential application for this technology is colorectal cancer screening. For example, inflammatory bowel disease (IBD) is a group of disorders characterized by chronic inflammation of the digestive tract wall. IBD is a serious, incurable, chronic condition, which can result in a higher risk for colorectal cancer. More frequent colonoscopy exam is recommended for people who live with IBD after 8-10 years. This technology will potentially improve the efficiency of detecting pre-cancerous lesions in people with IBD.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a spray cap for use on an endoscope according to the principles of the present teachings;

FIG. 2 is an end view of an endoscope;

FIG. 3 is a perspective view of a spray cap having a single oval slit orifice system system according to the principles of the present teachings;

FIG. 4 is a perspective view of a spray cap having a single cylindrical hole orifice system according to the principles of the present teachings;

FIG. 5 is a cross-sectional view of a spray cap and orifice system according to the principles of the present teachings; and

FIG. 6 is a perspective view of a spray cap having an inwardly tapered nozzle orifice system according to the principles of the present teachings;

FIG. 7 is a perspective view of a spray cap having an outwardly tapered nozzle orifice system according to the principles of the present teachings;

FIG. 8 is a perspective view of a spray cap having a centrally disposed cone member aligned with the orifice system according to the principles of the present teachings;

FIG. 9 is a perspective view of a spray cap having an offset tapered member aligned with the orifice system according to the principles of the present teachings;

FIG. 10 is a perspective view of a spray cap having fenestrated cover over the orifice system according to the principles of the present teachings;

FIG. 11 is a perspective view of a spray cap having a spiral member disposed within the orifice system with a central support according to the principles of the present teachings;

FIG. 12 is a perspective view, with portions removed for clarity, of the spray cap having the spiral member disposed within the orifice system according to the principles of the present teachings;

FIG. 13 is a perspective view of a spray cap having a spiral member disposed within the orifice system and coupled to the sidewalls of the orifice system according to the principles of the present teachings;

FIG. 14 is a perspective view, with portions removed for clarity, of the spray cap having the spiral member disposed within the orifice system and coupled to the sidewalls of the orifice system according to the principles of the present teachings; and

FIG. 15 is a perspective view of a spray cap having an offset member aligned with the orifice system according to the principles of the present teachings.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Inflammatory bowel diseases (IBD) affect approximately 1.5 million Americans. Due to repeat cycles of inflammation, patients are at increased risk of colon cancer over time. After 8 years, multiple gastrointestinal societies recommend colonoscopy every 1-2 years for evaluation of pre-cancerous changes. Unlike typical colon cancers, precancerous changes in patients with IBD are typically flat and therefore harder to detect using regular surveillance techniques.

Recent changes to the guidelines suggest the use of chromoendoscopy for cancer surveillance. This technique involves spraying the colon with a liquid dye that allows flat lesions to be highlighted and easily detectable. The dye is infused into the colon through the endoscope, but due to gravity and flow rate limitations of the fluid infuser, the colon is not uniformly and efficiently coated with dye using conventional systems.

To address this issue, device manufacturers have created a spray catheter, which is placed into an open channel of the endoscope, allowing the liquid to be sprayed more evenly across the colon. This, however, results in a significant limitation—there is only one instrument channel in an endoscope (the open channel or biopsy channel) and using this channel for the catheter prevents the use of this channel for other instrumentation via this channel. For example, if this channel needs to be used to biopsy the area, the spray catheter needs to be removed first and then later replaced. This is a time consuming process and messy.

To solve these issues, as illustrated in the figures, the present teachings provide a spray cap, which can be placed at the end of the endoscope, having a novel design and method of use. Generally, the spray cap provides partial coverage of the endoscope tip and, when placed correctly on the endoscope tip, will cover the irrigation channel but not the open channel or other operational features of the endoscope.

It should be understood that it is a unique challenge to break up, disperse, and/or atomize a relatively low-pressure stream of fluid as is used in endoscope applications. By way of example, conventional endoscopes can provide a fluid flow of about 3 mL/s at about 4-7 psi of pressure. A stream of relatively low pressure fluid does not want to “atomize” or disperse due to surface tension. It takes a relatively aggressive intervention to create a spray. A deficiency of existing spray catheters is that they often fail to achieve a true “spray” and at lower pressures simply provide a “disturbed” stream of liquid. If one could simply use higher pressures, this might not be such a challenge, but the relatively low pressures available means that the geometry becomes critical to success, as will be described and illustrated herein.

With reference to the figures, a spray cap 10 will be described for use on an endoscope 100 according to the principles of the present teachings. It should be recognized that the spray cap 10 can include any one of a number of variations and features, which are considered to be within the scope of the present teachings. Spray cap 10 is configured for use to be disposed on a distal end 102 of endoscope 100. Endoscope 100 can be a conventional endoscope having a plurality of channels and features for operation. In some configurations, endoscope 100 can comprise a centrally disposed optical lens 104 used for transmission of images to a clinician's display or monitor. To illuminate the area for imagining, endoscope 100 can further comprise one or more illumination sources (e.g. LED lights) 106 radially disposed adjacent optical lens 104. Still further, endoscope 100 can comprise an open channel 108 (e.g. biopsy channel) disposed adjacent to optical lens 104 to permit access to a patient's colon, for example. The open channel 108 can be used for introduction of any instruction, such as a biopsy instrument, during a medical procedure. Finally, endoscope 100 can comprise a liquid channel 110 for fluidly transmitting a liquid to distal tip 102 of endoscope 100. As described herein, liquid channel 110 can be used to infuse a dye into a colon of a patient for gastrointestinal evaluation and/or treatment. Liquid is introduced via liquid channel 110 under pressure.

To improve distribution of the liquid from liquid channel 110 and ensure proper coverage of liquid within the colon of the patient, spray cap 10 is provided. In some embodiments, spray cap or cap member 10 is formed as an integral member. In some embodiments, spray cap 10 can comprise a generally cylindrical body portion having downward projecting sidewalls 12 and an end wall 14 disposed on a distal end 16 of spray cap 10 and an opening 18 disposed on proximal end 20. Spray cap 10 can be selectively (e.g. removably) mounted to distal end 102 of endoscope 100 by insertion of distal end 102 of endoscope being inserted within opening 18 of the body portion of spray cap 10 and retained according to any reliable means, such as, but not limited to, press fit, friction fit, screw fit, retention fit, or other means sufficient to reliably retain spray cap 10 on endoscope 100 and permit removal thereof.

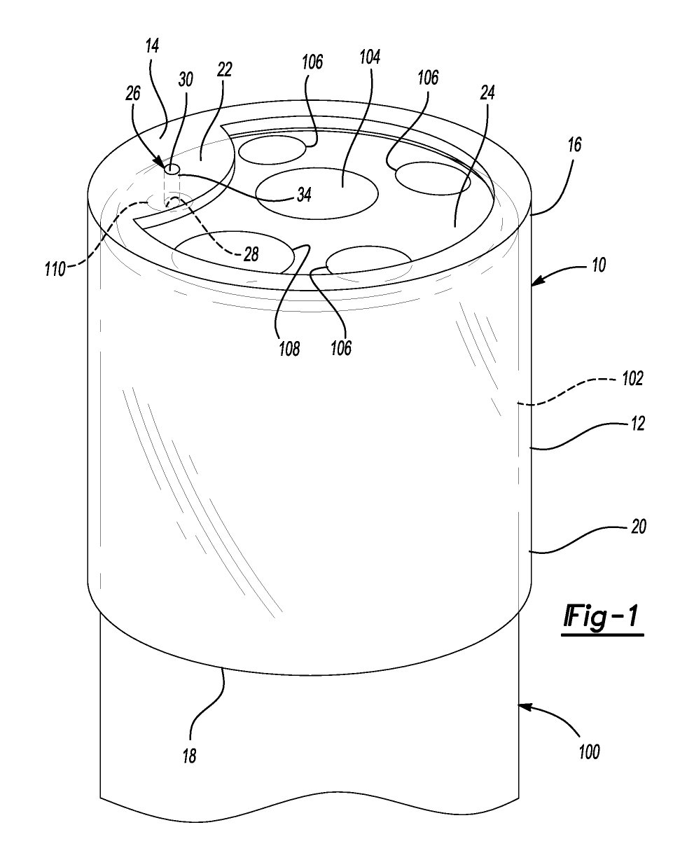

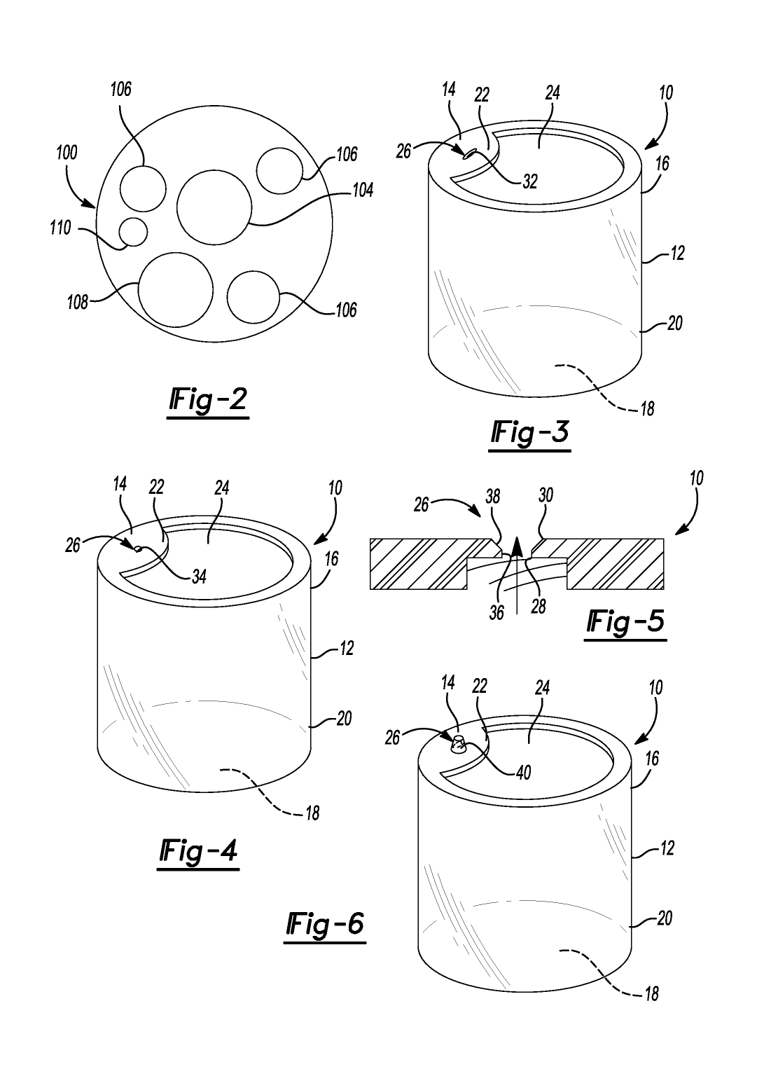

With particular reference to FIGS. 1 and 3-14, in some embodiments, end wall 14 of spray cap 10 can be formed to provide an obstruction portion 22 and an open portion 24. Open portion 24 can be sized and shaped to permit operation of optical lens 104, illumination sources 106, and/or open channel 108. To this end, it should be appreciated that open portion 24 can be configured to simply be optically “open”—that is, in some embodiments, open portion 24 can comprise a physically solid features that are optically transparent to enable illumination from illumination sources 106 to pass therethrough and optical lens 104 to remain optically functional. This can provide physical protection of illumination sources 106 and optical lens 104, without impeding their performance. However, if a physical opening is necessary, such as in connection with open channel 108, open portion 24 can remain physically clear to permit a tool or other feature to physically pass through end wall 14 of spray cap 10. In the illustrated embodiment, open portion 24 is both optically and physically open adjacent optical lens 104, illumination sources 106, and/or open channel 108.

Conversely, obstruction portion 22 is sized and shaped to overlay liquid channel 110 to obstruct liquid channel 110 and thereby urge pressurized liquid with liquid channel 110 through an orifice system 26. Orifice system 26 is a channel, through hole, or port extending through obstruction portion 22 of spray cap 10 that includes a first end 28 adjacent liquid channel 110, and in some embodiments fluidly coupled and sealed to liquid channel 110, and a second end 30 on an exposed end of spray cap 10. As will be described herein, orifice system 26 is operable for directing or otherwise affecting a flow of liquid exiting liquid channel 110 of endoscope 100 to enhance proper flow and/or distribution of liquid within a treatment volume (e.g. patient colon).

In some embodiments, as illustrated in FIGS. 3 and 4, orifice system 26 can comprise a small slit or elongated (e.g. rectangular, oval, etc.) fenestration 32 (FIG. 3) or generally circular hole or fenestration 34 (FIG. 4) formed through obstruction portion 22 of spray cap 10. The slit 32 can be sized and shaped to modify the initial flow path of the liquid and provide a resultant fan shaped distribution of liquid during injection. Likewise, hole 34 can be sized and shaped to provide a generally uniform circular distribution of liquid during injection. It should be understood that slit 32 and/or hole 34 can define any size or shape necessary to achieve a desired flow pattern. Slit 32 and hole 34 are particularly simple to manufacture during convention manufacturing techniques, such as injection molding, drilling, and the like.

With particular reference to FIG. 5, it should be understood than orifice system 26 can comprise a non-cylindrical cross-section, such as a tapered profile, and/or can include a combination of a first sidewall 36 parallel to a flow direction and second sidewall tapered 38 relative to the flow direction.

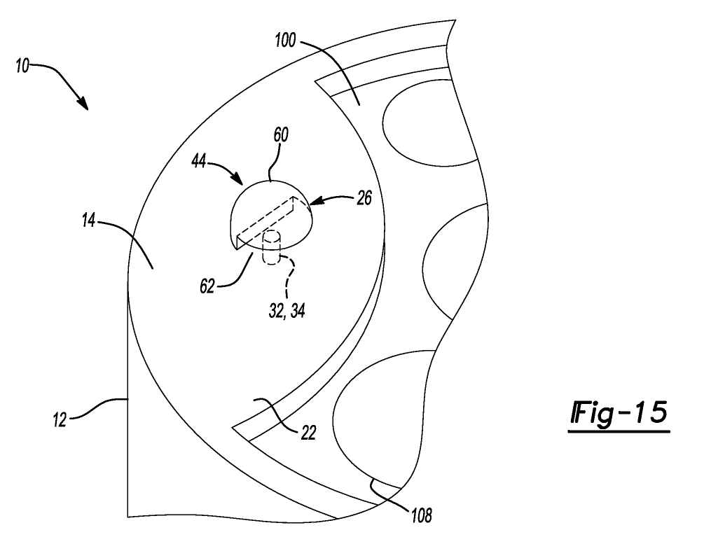

Referring now to FIGS. 6-14, in some embodiments, orifice system 26 can comprise any one of a number of flow restrictor and/or nozzle shapes operable to provide a spray pattern of liquid conducive to a particular medical procedure. It should be understood that although the following embodiments will be illustrated and described as being raised above a surface of obstruction portion 22, the following embodiments can be formed such that orifice system 26 is fully contained within and/or flush with the surface of obstruction portion 22. In other words, in some embodiments, the orifice system 26 and the following nozzle features can stand above obstruction portion 22, flush with obstruction portion 22 (FIG. 15), and/or submerged relative to an outer surface of obstruction portion 22. From a fluidic standpoint, the standoff orientation in the several drawings is not required.

In some embodiments, as illustrated in FIG. 6, orifice system 26 can define an inwardly tapered profile 40, relative to a flow direction, conducive for forming a direction spray pattern. However, in some embodiments, as illustrated in FIG. 7, orifice system 26 can define an outwardly tapered profile 42, relative to the flow direction, conducive for forming a generally circular, radial spray pattern.

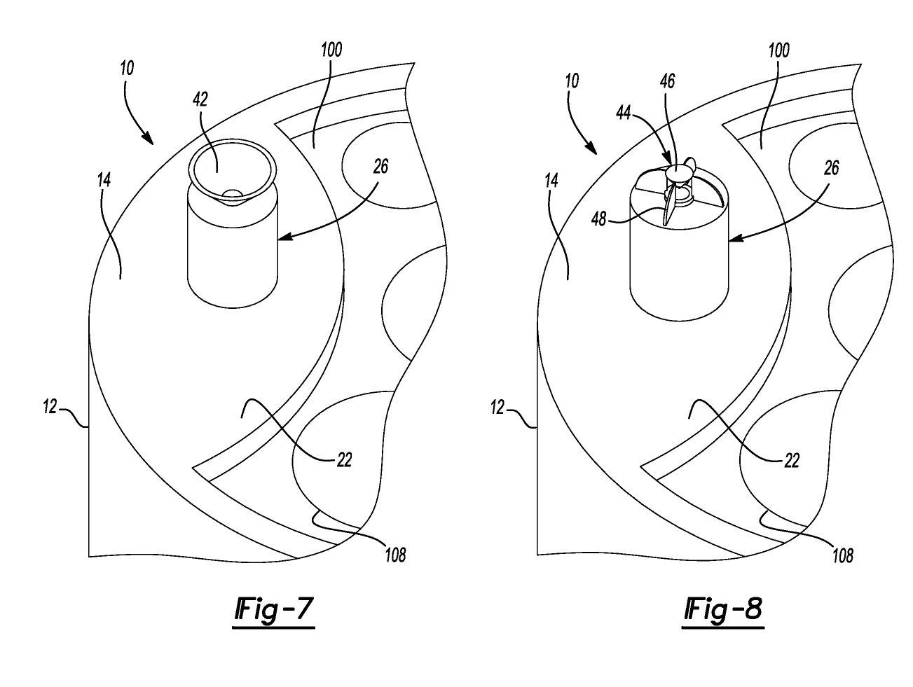

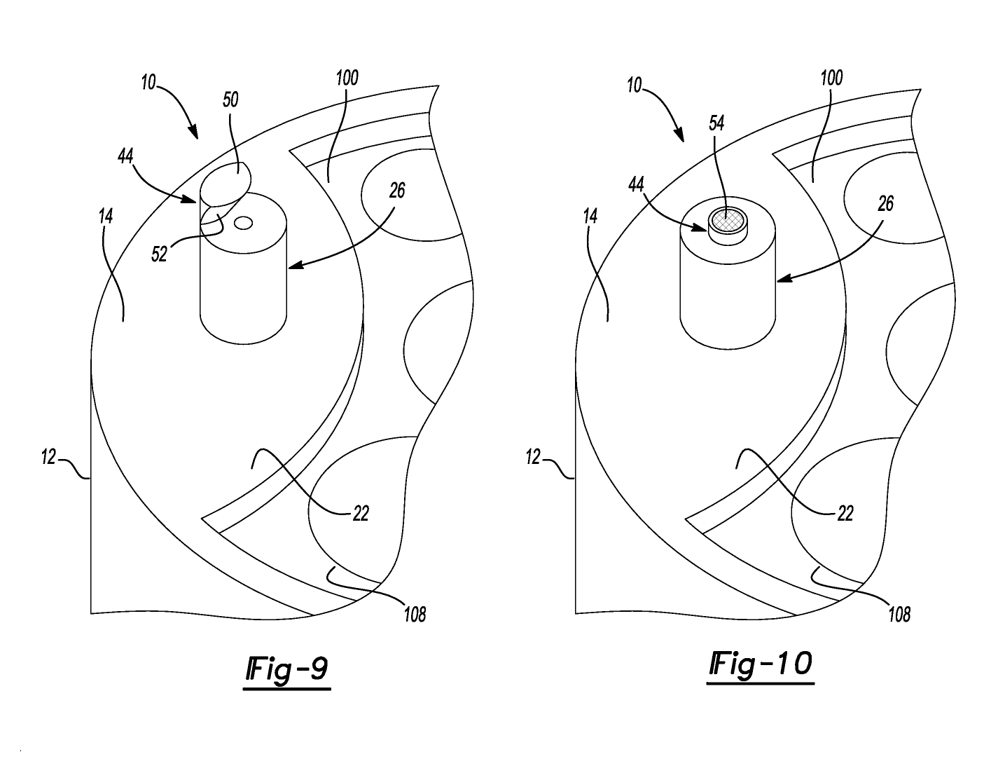

In some embodiments, as illustrated in FIGS. 8-14, orifice system 26 can comprise a deflection member 44 disposed within a flow path of the liquid. In some embodiments, deflection member 44, as illustrated in FIG. 8, can comprise a centrally disposed cone member 46 being centrally aligned with the flow path of the liquid. The centrally disposed cone member 46 can be positioned having a cone tip generally central within the flow path of the liquid and retained in position by a plurality of cone support members 48 radially extending outwardly and in connection with cone member 46. The cone member 46 can be operable to direct the flow of the liquid outwardly, thereby ensure enhanced radial coverage. In some embodiments, as illustrated in FIG. 9, deflection member 44 can comprise an offset tapered member 50 being disposed to a side of orifice system 26, thereby interrupting the flow path of the liquid and resulting in deflection of the liquid to an opposing side. In some embodiments, offset tapered member 50 can comprise an arcuate impact surface 52 shaped to smoothly deflect the flow path to an offset fan-like pattern. The offset tapered member 50 can be disposed on an outboard side of orifice system 26 relative to a central axis of endoscope 100, thereby serving to deflect the liquid to a central location relative to endoscope 100 and, consequently, the treatment volume.

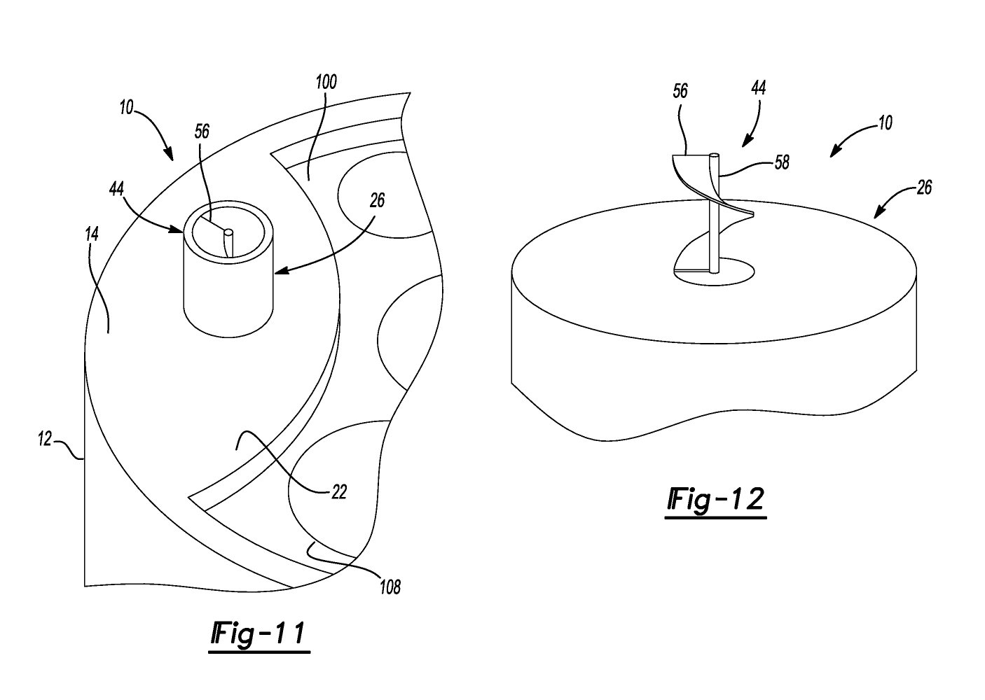

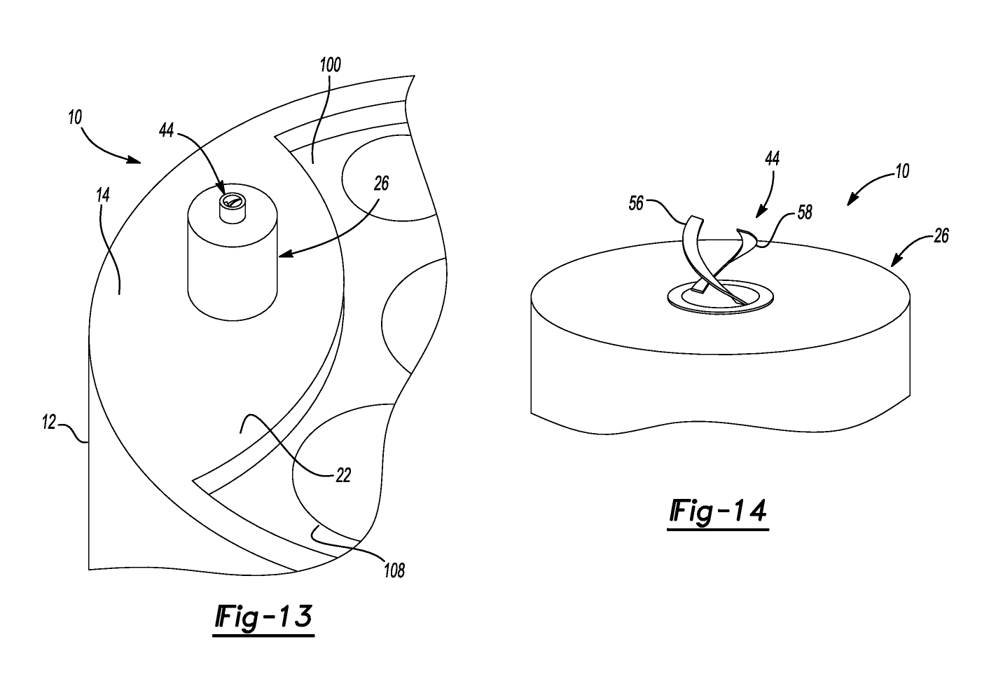

In some embodiments, as illustrated in FIG. 10, deflection member 44 can comprise a screen or fenestrated cover 54 over orifice system 26 resulting in a modified flow path of liquid in the form of a distributed spray or mist of liquid. Similarly, as illustrated in combination FIGS. 11 and 12, and FIGS. 13 and 14 (note that FIGS. 12 and 14 illustrate the internal structure of orifice system 26 with sidewalls removed from clarity), deflection member 44 can comprise a spiral member 56 being disposed within orifice system 26 to urge the liquid in a rotational direction, thereby creating a rotational distribution pattern within the treatment volume. In some embodiments, such as illustrated in FIGS. 11 and 12, spiral member 56 can be anchored to a centrally disposed post member 58 extending co-axially with the flow path of the liquid. Spiral member 56 can comprise a generally flat plate surface rotationally extending about centrally disposed post member 58. Similarly, in some embodiments, as illustrated in FIGS. 13 and 14, spiral member 56 can be anchored to a sidewall of orifice system 26 and form a helix design.

In some embodiments, as illustrated in FIG. 15, deflection member 44 can comprise a dome shaped member 60 (or other shape) being disposed over hole 32 or 34 to a slit or gap 62 (exaggerated in size to show detail). Slit or gap 62 can be a small sized slit to interrupt the fluid flow from hole 32, 34 and disperse and/or atomize the liquid. It should be understood that the gap 62 can be project to any particular direction as desired to achieve a predetermined fluid distribution pattern. It should also be understood that member 60 can define any one of a number of impact surfaces 64 to further tailor the fluid distribution, such as, but not limited to, an arcuate surface, a cone surface, or other shape.

It should be understood that although several embodiments have been disclosed having varying shapes, sizes, contours, and methods of operation, the features and teachings of these embodiments can be combined in any combination to achieve a desired fluid distribution profile.

According to the principles of the present teachings, spray cap 10 thus provides more uniform coating of the colon wall when liquid dye is sprayed. Spray cap 10 thus permits uniform application of dye throughout the colon, a more efficient modality of application, and does not require obstruction of the single open channel thereby permitting additional instrumentation.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A spray cap for use with an endoscope, said endoscope having a liquid channel and an open channel, the liquid channel being configured to permit a liquid to flow along and exit the liquid channel along an initial flow path, said spray cap comprising:
a body portion connectable with an endoscope, the body portion having an end wall having an obstruction portion being configured to be positioned over the liquid channel of the endoscope and an open portion being configured to be positioned over the open channel; and
an orifice system extending through the obstruction portion of the end wall and in fluid communication with the liquid channel of the endoscope to permit liquid from the liquid channel of the endoscope to flow through the orifice system and into a treatment volume, said orifice system being spaced apart from the open channel to permit unimpeded use of the open channel, said orifice system being configured to obstruct the liquid channel and result in a modified flow pattern of the liquid from the liquid channel of the endoscope.

2. The spray cap according to claim 1 wherein said orifice system is a flat elongated fenestration.

3. The spray cap according to claim 1 wherein said orifice system is a single oval fenestration.

4. The spray cap according to claim 1 wherein said orifice system comprises an inwardly tapered surface.

5. The spray cap according to claim 1 wherein said orifice system comprises an outwardly tapered surface.

6. The spray cap according to claim 1, wherein said orifice system comprises a deflection member disposed in the initial flow path of the liquid to obstruct the initial flow path and result in the modified flow path.

7. The spray cap according to claim 6 wherein the deflection member is a centrally disposed cone member being supported in the initial flow path via a plurality of cone support members.

8. The spray cap according to claim 6 wherein the deflection member is an offset tapered member being supported to a side of the initial flow path.

9. The spray cap according to claim 6 wherein the deflection member is a fenestrated cover.

10. The spray cap according to claim 6 wherein the deflection member is a spiral member anchored to a centrally disposed post member.

11. The spray cap according to claim 6 wherein the deflection member is a spiral member anchored to a sidewall of said orifice system.
